(19) United States
(12) Patent Application Publication
(10) Pub. No.: US 2018/0326200 A1
(43) Pub. Date: Nov. 15, 2018

(54) MULTI-WAY VALVE WITH MULTIPLE RETENTION RING

(71) Applicant: Hans-Juergen HOPF, Zirndorf (DE)

(72) Inventors: Michael HOPF, Zirndorf (DE); Alexander HOPF, Nuernberg (DE); Norbert KASSEL, Oberasbach (DE)

(21) Appl. No.: 15/776,671

(22) PCT Filed: Nov. 3, 2016
(86) PCT No.: PCT/EP2016/076586

§ 371 Date: May 16, 2018

(30) Foreign Application Priority Data
Nov. 17, 2015 (DE) 10 2015 119 899.1

(51) Int. Cl. A61M 39/22; F16K 11/085; F16K 27/06

(52) U.S. Cl. CPC A61M 39/223; A61M 2039/229; F16K 27/065; F16K 11/0853

(57) ABSTRACT

A multi-way valve including at least one main housing through parts of which a medium can flow, and an actuator which is accommodated in the main housing to be rotatable about a central axis. The main housing has at least two connection points to admit and discharge the medium and forms an actuator receptacle which has a peg that is concentric to the central axis. The actuator includes a control element and a substantially cylindrical section which accommodates at least a portion of the peg and has at least one through-hole to fluidically connect at least two connection points. The peg has at least one abutment for the actuator such that a sealing surface is formed in the region of at least one of the connection points. Protrusions and recesses are arranged to correspond to each other and which jointly form a joint between the actuator and the actuator receptacle.

The present invention relates to a multi-way valve, in particular 2-, 3- and 4-way valves as used, in particular in medicine.

Multi-way valves, in particular 2-, 3- and 4-way valves are known in the prior art and are used in particular in medicine and medical technology. Here they are used, in particular, in the field of infusions, in the field of artificial feeding, in the transfusion and in particular the supply/transfer of various through-flow media and as so-called injection equipment for medical and pharmaceutical equipment. The multi-way valves are also used in medical systems which consist of multiple components. Such systems include gravity infusions, pumps or pump transfer systems, tube feeding systems, injections, combinations therefore and suchlike.

Furthermore, through combination with several multi-way valves, a multi-way valve can be assembled into a multi-way valve bank or manifold (multiple distributor).

When providing multi-way valves a predetermined tightness, ease of operation and absence of burrs are important. In particular, it should be ensured that multi-way valves for supplying different media which also have very different viscosities are used.

One of the drawbacks of the multi-way valves known in the prior art is that they exhibit weaknesses in various cases of applications including in terms of tightness, user-friendliness and problems with the flow of media of different viscosities.

The aim of the present invention is to at least partially overcome the known disadvantages in the prior art. This task is solved by an invented multi-way valve according to claim 1. Preferred forms of embodiment of the multi-way valve are the subject matter of the respective sub-claims.

The multi-way valve in accordance with the invention for use in medicine and/or medical technology comprises at least one main housing, through parts of which a medium can flow, and an actuator which is accommodated in said main housing in such a way as to be rotatable about a central axis. The multi-way valve in accordance with the invention is characterised in that it has connection points for admitting and discharging the medium.

Moreover, the main housing of the multi-way valve according to the invention forms an actuator receptacle which is characterised in that it has a peg that is arranged concentrically to a central axis of the main housing. The actuator of the multi-way valve according to the invention has a control element and an essentially cylindrical section which accommodates at least a portion of the peg of the actuator receptacle of the main housing and also has a through-hole for fluidically connecting at least two connection points.

The peg of the main housing of the multi-way valve according to the invention is designed so that in the region of one connection point of the main housing it has or provides an abutment for the actuator, which together with the actuator forms a sealing surface in the region of at least one connection point. The multi-way valve according to the invention is characterised in that the multi-way valve has protrusions and recesses which are arranged so as to correspond with each other and together form a joint connection been the actuator and actuator receptacle and in that at least two joint connections are provided, wherein at least one protrusion and/or recess is arranged so as to extent radially about the inner side of the main housing of the actuator.

In accordance with the present invention the term medium is taken to mean all types of fluid, in particular liquids, such as, for example, aqueous liquids, blood, nutritional solutions etc. and also gases.

In accordance with the present invention the term main housing is taken to mean a component of the multi-way valve which comprises the housing with the connection points and the actuator receptacle, wherein the latter is taken to mean the region of the main housing used for accommodating the actuator.

In accordance with the present invention the term actuator is taken to mean at least one so-called “plug” that closes the flow opening. The control element in accordance with the present invention is an element firmly attached to the actuator, for example a tap, with which the actuator can be rotated about the central axis. This can comprise one or more, usually two, three or four actuating levers.

Understood as an abutment in accordance with this present invention is an area of the actuator receptacle which takes up the pressure on the actuator, in particular in the closed state, or counters deformation of the actuator under the pressure of the medium.

The modification of the actuator and actuator receptacle through the increase in the number of protrusions and recesses corresponding to each other in accordance with the invention and forming at least two joint connections between the actuator and actuator receptacle results in greater pull-out resistance between the actuator and actuator receptacle. This ensures that the tap cannot be displaced from its defined, set position by the used, in particular viscous, medium and leakage of the used, in particular viscous, medium is fully or almost fully prevented.

In accordance with a preferred form of embodiment of the multi-way valve according to the invention the protrusions and recesses are each arranged on the actuator/the actuator receptacle corresponding to each other in pairs in a sequence of two recesses and then two protrusions or, vice versa, in a sequence of two protrusions and then two recesses (i.e. in a 2+2 pattern). In a further preferred form of embodiment of the multi-way valve in accordance with the invention the recesses are arranged on the actuator/actuator receptacle corresponding to each other in pairs and the protrusions singly in a sequence of two recesses and then one protrusion or in a sequence of one protrusion and then two recesses or in a sequence of one protrusion and then two recesses followed by a protrusion (i.e. in a 2+1, 1+2 or 1+2+1 pattern).

According to a further preferred form of embodiment the protrusions are arranged on the actuator/actuator receptacle corresponding to each other in pairs and the recesses singly in a sequence of two protrusions and then one recess or in a sequence of one recess and then two protrusions or in a sequence of one recess and then two protrusions followed by a recess (i.e. in a 2+1, 1+2 or 1+2+1 pattern). In a further preferred form of embodiment of the multi-way valve according to the invention, the recesses are arranged on the actuator/actuator receptacle corresponding to each other in groups of three and the protrusions singly in a sequence of three recesses and then one protrusion or in a sequence of one protrusion and then three recesses (i.e. in a 3+1 or 1+3 pattern). In a further preferred form of embodiment of the multi-way valve according to the invention, the protrusions are arranged on the actuator/actuator receptacle corresponding to each other in groups of three and the recesses singly in a sequence of three protrusions and then one recess or in a sequence of one recess and then three protrusions (i.e. in a 3+1 or 1+3 pattern). Through the arrangement of the recesses and protrusions of the aforementioned forms of embodiment in the form of a labyrinth seal, in addition to better mechanical fixation better sealing of the arranged components is also achieved.

According to a further preferred form of embodiment of the multi-way valve according to the invention, all the recesses are arranged on the actuator and/or all the protrusions are arranged on the actuator receptacle, or according to a further preferred form of embodiment the recesses and the protrusions are arranged alternately on the actuator and the actuator receptacle each corresponding with each other.

According to another, particularly preferred form of embodiment, in the case of the actuator receptacle the diameter of the recesses and/or the diameter of the protrusions gradually becomes smaller in the direction of the interior of the actuator receptacle so that the innermost recess and/or the innermost protrusion has the smallest diameter of the present recesses and/or protrusions and the diameter of every additionally present recess and/or protrusion then gradually increases in the direction of the surface of the actuator receptacle so that the recess and/or protrusion on the outermost edge has the largest diameter of the present recesses and/or protrusions. In the same way in the case of the actuator the diameter of the recesses and/or protrusions gradually becomes smaller in the direction of the cylindrical section of the actuator so that the innermost recesses and/or the innermost protrusion has the smallest diameter of the present recesses and protrusions and the diameter of each additionally present recess and/or protrusion then gradually increases in the direction of the control element of the actuator so that the recess and/or protrusion nearest the control element has the largest diameter of the present recesses and/or protrusions. This gradual design ensures that when assembling the multi-way valve according to the inventions, on lowering the actuator into the actuator receptacle the protrusions and corresponding recesses of the outer and inner joint connections simultaneously engage flush and only in envisaged joint connection positions, i.e. without the inner recesses and/or protrusions of the actuator engaging in another recess and/or protrusion of the actuator before they have assumed their final position within the multi-way valve.

In a further form of embodiment of the multi-way valve according to the invention, the main housing comprises two, three or four connection points.

According to a further, particularly preferred form of embodiment, the medium flowing through the multi-way valve is a liquid, more particularly selected from a group which includes solutions for injection, solutions for infusion, nutritional solutions, blood, plasma as well as combinations thereof and suchlike.

According to a further, particularly preferred form of embodiment the fluid flowing through the multi-way valve has a viscosity of 0.7 mPa (s⁻¹) and 10⁶ mPa (s⁻¹), preferably between 1 mPa (s⁻¹) and 10⁵ mPa (s⁻¹) and particularly preferably around 10² mPa (s⁻¹).

According to a further form of embodiment of the multi-way valve in accordance with the invention the peg of the main casing is a conical, at least in parts. In a further particularly preferred form of embodiment conical and non-conical sections and/or a conical section with different cone angles are provided in stepped sequence. In the present situation the cone angle is taken to mean the deviation from the central axis; in particular, positive and negative angles are understood as cone angles. In a further, particularly preferred form of embodiment the peg has an alternating sequence of positive and negative cone angles. In a further preferred form of embodiment the peg has, in particular, conical section offset inward and outwards relative to the central axis.

According to a further preferred form of embodiment the inner side of the cylindrical section is conical at least in parts and is essentially adapted to the conical course of the peg.

According to the present invention the through openings of the actuator are gate-like, more particularly designed so that the through opening is open toward the end of the cylindrical section of the actuator. Furthermore, in particular, the size of the through opening is preferably adapted depending on the fluid to be used, wherein a combination of the shape of the abutment, for example conical and rectangular, is also taken into consideration.

According to a further, particularly preferred form of embodiment of the multi-way valve in accordance with the invention, the number of through openings of the actuator corresponds to the number of connection points on the main housing. Thus, for example, a form of embodiment with two connection points has two through openings. According to a further preferred form of embodiment the actuator has fewer through openings than there are connection points on the main casing, wherein at least one and in particular two through openings or through openings designed as gate are provided.

In accordance with a further particularly preferred form of embodiment of the multi-way valve according to the present invention, each joint connection has a radially surrounding sealing section and/or a positioning section. In particular, the at least two joint connections between the actuator and the main housing of the actuator receptacle are designed in such a way that that through the connections between the two parts a sealing in the region of more than 3 bars to more than 8 bars is achieved.

This allows the multi-way valve according to the invention also to be used for viscous fluid such as feeding solution (e.g. for tube feeding systems), in the case of the use of which, due to the higher medium viscosity, higher pressures occur which attack the joint connections of the multi-way valve, without leak, with leak reinforcements.

According to a further preferred form of embodiment the protrusions on the actuator and/or on the actuator receptacle have a partially rounded shape and the corresponding recesses on the actuator receptacle and/or actuator are adapted in shape for the form-fitting receiving of the corresponding partially rounded protrusions and for forming the respective joint connection.

According to a further particularly preferred form of embodiment of the multi-way valve in accordance with the present invention the cylindrical section of the actuator is at least in parts made of solid material. More particularly the cylindrical section of the actuator is made of solid material in the region of the joint connection. Through this, compared with a hollow cylinder embodiment greater dimensional stability and material strength is achieved in the section in question which there makes for improved pull-off resistance between the actuator and the actuator receptacle so as to ensure that leakage of thus used, in particular, viscous, medium is fully or almost fully prevented. In particular, in this way a pull-off resistance of more than 80 N, preferably more than 150 N, particularly preferably more than 200 N and preferred most of all more than 250 N is achieved. Preferably a pull-off resistance in the region of more than 150 N to more than 250 N is achieved.

According to a further preferred form of embodiment of the multi-way valve in accordance with the invention at least the housing and/or the actuator are at least made of a material that is selected from a group which in addition to copolyester includes further materials such as, for example, duroplastic and thermoplastic plastic and in particular polyphenyl sulphide, polypropylene, poly-1-butene, polyvinylchloride, polyvinylidene chloride, polymethyl meta-acrylate, polyacryl nitrile, polystyrene, polysulfone. polyacetal, polyvinyl alcohol, polyvinyl acetate, ionomers, fluorine plastics, polyethylene, polyamide, more particularly partially aromatic polyamide, polycarbonate, polyester, copolyester, polyphenylene oxide, polysulfone. polyvinyl acetal, polyurethane and chlorinate polyether, cellulose nitrate, cellulose acetate, cellulose ether, phenol resin, urea resin, thiourea resin, melamine resin, alykyl resin, allyl resin, silicone, polyimide, polybenzimidazole, epoxide resin, casein plastic, crosslinked polyurethane, polyvinyl chloride, unsaturated polyester resin, antimicrobial or antiseptic materials such as, for example, highly porous silver, ion-free silver, silver compounds and in particular micro-silver, compounds releasing metal ions and materials containing no softening agent, in particular not bisphenol A or phthalates, as well as combinations thereof and suchlike.

In accordance with a further preferred form of embodiment, the multi-way valve according to the invention is characterised in that at least the surfaces of the connection system which come into contact with the fluid, in particular the inner surfaces of the multi-way valve, are at least in sections made of a material which has antiseptic and/or antimicrobial properties. Such antimicrobial or antiseptic materials are, for example, materials such as highly porous silver, ion-free silver, silver compounds and in particular micro-silver, compounds releasing metal ions, combinations thereof and suchlike. Preferably these are arranged in the region of the respective surfaces, wherein in accordance with a specially preferred form of embodiment the relevant materials are also incorporated into the plastic of which the multi-way valve or its component are made. Thus, in particular, a highly porous silver which is preferably also essentially produced in an ion-free manner can be mixed into the plastic, wherein at least part of the components of the multi-way valve are then made of this. Alternatively it is also within the scope of the present invention for the surfaces to be coated with a corresponding material or material combination.

In accordance with further particularly preferred form of embodiment of the present invention an amorphous copolyester is used for the production of the multi-way valve for medicine and medical technology and its use for infusion or transfusion lines, multi-way valves, multiple distributors, injection equipment such as needles, accesses or suchlike and combinations thereof. The system is characterised in that at least the surfaces of the connection system that come into contact with the fluid are at least in parts made of an amorphous copolyester.

According to a further, particularly preferred form of embodiment, at least in parts materials are used for the production of the connection system according to the invention which do not contain softening agents, in particular no phthalates and no bisphenol A.

The present invention also includes the use the multi-way valve according to the invention in medicine and medical technology, in particular for the admission and transfer of various flow media, in particular for gravity infusion, for pump transfer systems, feeding tube systems, injections as well as combinations thereof and suchlike.

The invention will be explained below by way of a preferred example of embodiment, whereby it is pointed out that through this example modifications or additions as immediately evident to a person skilled in the art are also included. In addition, this preferred example of embodiment does not represent a restriction of the invention in the manner that modifications and additions are within the scope of the present invention.

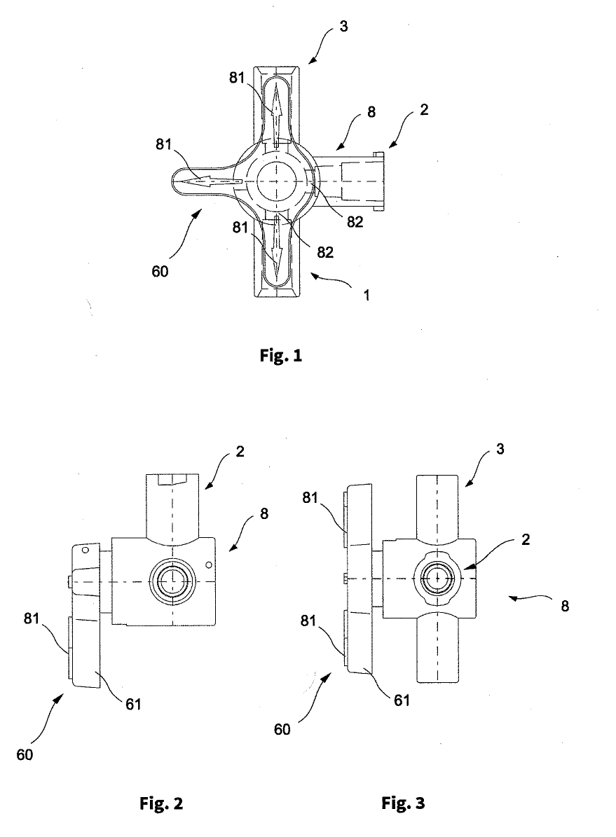

FIGS. 1 to 3 show a view from and above and a side view of a particularly preferred form of embodiment of a multi-way valve;

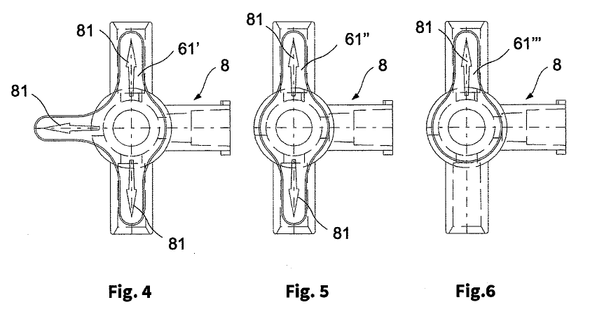

FIGS. 4 to 6 show a view from above of differently designed multi-way valves;

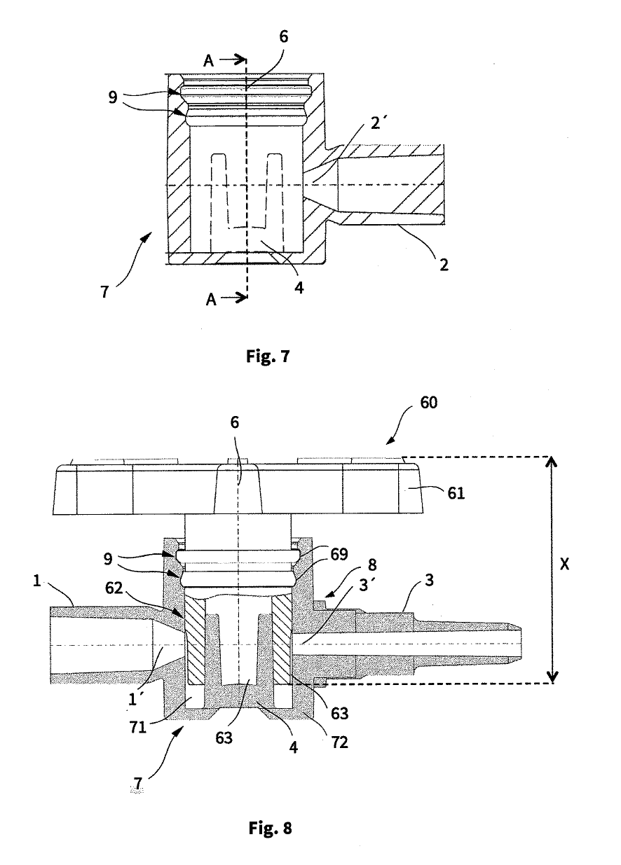

FIG. 7 shows a cross-section through the main housing of a three-way valve according to the invention; and

FIG. 8 shows a cross-section through the main housing of an invented three-way valve according to FIG. 7 with an inserted actuator.

FIGS. 1 to 3 each show a view from above in FIG. 1 and a side view in each of FIGS. 1 and 3 of a form of embodiment of a multi-way valve in accordance with the invention. FIG. 1 shows the main housing 8 with three connection points 1, 2, 3. The actuator 60 has a control element 61 in the form of a tap with three control levers which is firmly connected to a cylindrical section 62 (not shown). The cylindrical section 62 is arranged concentrically about a central axis 6 (not shown). At the end of the cylindrical section 62 opposite the control element there are gate-shaped through openings 82 (gates). The form of embodiment shown here shows an actuator 60 in which in the cylindrical section 62 only two through openings 82 (gates) are provided which are arranged at an angle of 90° to each other. In accordance with this arrangement, depending on the switching, connections 1 and 2 or 2 and 3 are fluidically connected. A connection between connection 3 and 1 is not possible.

FIGS. 4 to 6 each show a view from above of differently designed multi-way valves in which, in particular, the control elements 61′, 61″, 61″′ are differently designed. FIG. 4 thus shows a three-arm control element 61′, FIG. 5 shows a two-arm control element 61″ and FIG. 6 shows a one-arm control element 61″′ as can be used in combination with a differently designed main housing 8. The arrows 81 applied to the control element are also for easier orientation when using the multi-way valve in practice, in order to schematically indicate the flow direction of the fluid.

The example of embodiment according to FIG. 7 shows a cross-section through the main housing 8 (shown with rough hatching) with two recesses 9 on the inside of the actuator receptacle 7 radially extending about the central axis 6, wherein each recess 9 is designed in such a way that it has different depths. In the actuator receptacle 9 a peg 4 is arranged about a central axis 6. The connection point 2 is connected with the actuator receptacle wherein a through opening 2′ is formed. In this example of embodiment the inner recess 9 has a smaller diameter than the outer recess 9 and both recesses 9 have a shape adapted to the partially round shape of the protrusions 69 of the mountable actuator 60 (not shown, but cf. FIG. 8) in order to ensure form-fitting of the joint connection when assembling the multi-way valve according to the invention and to thereby increase tightness of the multi-way valve. The dashed lines marked A-A indicate the section line A-A for the cross-sections in FIG. 8.

FIG. 8 thus shows the connection of the main housing (shown with fine hatching) in the direction of the A-A line with the actuator 60 in accordance with FIG. 7 as one form of embodiment of the multi-way valve according to the invention. The actuator 60 is pressed up to a predetermined pressing-in depth X in to the actuator receptacle 7 of the main housing. In this position the two partially round protrusions 69 of the cylindrical section 62 of the actuator 60 engage in a form-fitting manner in the two recesses 9 on the inside of the actuator receptacle 7 of the main housing 8. The cylindrical section 62 (shows with rough hatching) of the actuator 60 encompasses the peg 4 of the actuator receptacle 7 arranged around a central axis 6 and thus forms an abutment for the actuator 60. In the open state of the multi-way valve the medium flows through from one of the connection points 1, 3, through the associated through opening of the main housing 1′, 3′ through a gate-shaped through opening 63 of the actuator 60 via free space 71 between the actuator 60 and the base 72 of the actuator receptacle 7 to another through opening 63 of the actuator and out of the other connection points 1, 3 of the main housing through the other through opening 1′, 3′ of the main housing. Sealing in the upper area is achieved through a defined press-in depth of the actuator 60 into the joint edge. In the inside of the main housing 8 sealing with regard to the corresponding through openings 1, 2, 3 is ensured by way of the conical abutment formed by the peg 4.

1. Multi-way valve for use in medicine or medical technology, the valve comprising:
at least one main housing through parts of which a medium can flow, and an actuator which is accommodated therein in such a way as to be rotatable about a central axis, wherein
the main housing has at least two connection points for admitting and discharging the medium and forms an actuator receptacle which has a peg that is concentric to the central axis and
the actuator has a control element and an essentially cylindrical section which accommodates at least a portion of the peg of the actuator receptacle and which has at least one through opening for fluidically connecting at least two connection points and
the peg of the actuator receptacle has at least one abutment for the actuator such that a sealing surface is formed in the region of the at least one connection point,
the multi-way valve has protrusions and recesses, which are arranged so as to correspond with each other and which together form a joint connection between the actuator (60) and actuator receptacle and in that at least two joint connections are provided,
wherein at least one of a protrusion and a recess is disposed so as to extend radially about the inner side of the main housing of the actuator and/or radially about the outer side of the cylindrical section of the actuator receptacle and wherein all recesses are arranged on the actuator and all protrusions are arranged on the actuator receptacle, or the recesses and protrusions are arranged in an alternating manner on the actuator and/or the actuator receptacle, and
the diameter of the recesses and/or the diameter of the protrusion becomes smaller in the direction of the interior of the actuator receptacle so that during assembly when lowering the actuator into the actuator receptacle, the recesses and the corresponding protrusions of the outer and inner joint connections simultaneously engage flush and only in the envisaged joint connection positions at a predetermined press-in depth.

2. (canceled)

3. Multi-way valve according to claim 1 wherein the main housing has two, three or four connection points.

4. Multi-way valve according to claim 1 wherein the medium is a fluid which is selected from a group which includes solutions for injection, solutions for infusion, nutritional solutions, blood, plasma, gases, and combinations thereof.

5. Multi-way valve according to claim 4 wherein fluid has a viscosity which is between 0.7 mPa (s⁻¹) and 10⁶ mPa (s⁻¹).

6. Multi-way valve according to claim 1 wherein the peg is at least partially conical.

7. Multi-way valve according to claim 1 wherein the inner side of the cylindrical section of the actuator is at least partially conical.

8. Multi-way valve according to claim 1 wherein the through openings of the actuator are designed as gates, which are open towards the end of the cylindrical section of the actuator.

9. Multi-way valve according to claim 1 wherein the number of through openings of the actuator corresponds to the number of connection points in the main housing.

10. Multi-way valve according to claim 1 wherein each joint connection has a radially extending sealing section and/or positioning section.

11. Multi-way valve according to claim 1 wherein the at least two joint connections between the actuator and the main housing of the actuator receptacle provide a sealing of greater than 3 bars.

12. Multi-way valve according to claim 1 wherein the cylindrical section of the actuator is made of a solid material at least in the region of the joint connection.

13. Multi-way valve according to claim 1 at least the main housing and/or the actuator are at least in parts made of a material selected from a group which in addition to copolyester, particular amorphous copolyester includes further materials such as, for example, duroplastic and thermoplastic plastic and in particular polyphenyl sulphide, polypropylene, poly-1-butene, polyvinylchloride, polyvinylidene chloride, polymethyl meta-acrylate, polyacryl nitrile, polystyrene, polysulfone. polyacetal, polyvinyl alcohol, polyvinyl acetate, ionomers, fluorine plastics, polyethylene, polyamide, more particularly partially aromatic polyamide, polycarbonate, polyester, copolyester, polyphenylene oxide, polysulfone. polyvinyl acetal, polyurethane and chlorinate polyether, cellulose nitrate, cellulose acetate, cellulose ether, phenol resin, urea resin, thiourea resin, melamine resin, alykyl resin, allyl resin, silicone, polyimide, polybenzimidazole, epoxide resin, casein plastic, crosslinked polyurethane, polyvinyl chloride, unsaturated polyester resin, antimicrobial or antiseptic materials such as, for example, highly porous silver, ion-free silver, silver compounds and in particular micro-silver, compounds releasing metal ions and materials containing no softening agent, in particular not bisphenol A or phthalates, as well as combinations thereof and suchlike.

14. (canceled)
